(19) United States
(12) Patent Application Publication
(10) Pub. No.: US 2014/0348916 A1
(43) Pub. Date: Nov. 27, 2014

(54) Treatment of Pain with Oral Dosage Forms Comprising Zoledronic Acid and An Enhancer

(71) Applicant: Antecip Bioventures II LLC, New York, NY (US)

(72) Inventor: Herriot Tabuteau, New York, NY (US)

(21) Appl. No.: 14/456,939

(22) Filed: Aug. 11, 2014

(51) Int. Cl. A61K 31/675; A61K 9/28; A61K 47/14

(52) U.S. Cl. CPC A61K 31/675; A61K 47/14; A61K 9/28
USPC 424/468; 514/94; 424/474

(57) ABSTRACT

Disclosed herein are methods of treating or preventing pain. Typically, a pharmaceutical composition having a therapeutically effective amount of the zoledronic acid is administered to a mammal suffering from pain. The pharmaceutical composition may further comprise an enhancer, which can be a medium chain fatty acid salt, an ester, an ether, or a derivative of a medium chain fatty acid and can have a carbon chain length of from about 4 to about 20 carbon atoms.

### FIELD

The present disclosure generally relates to the compositions of bisphosphonates and the methods of treating pain and medical conditions associated with pain by using pharmaceutical composition comprising a bisphosphonate compound.

### BACKGROUND

Bisphosphonates are an important class of drugs that have demonstrated promising effects in treating pain related to diseases associated with abnormally accelerated bone resorption such as, but not limited to, osteoporosis, Paget's disease, tumor induced hypercalcaemia and more recently, bone metastases and other related illnesses that are associated with painful conditions.

### SUMMARY

Disclosed herein are methods of treating or preventing pain caused by a medical condition in a subject, the method comprising: administering to the subject a pharmaceutical composition having a therapeutically effective amount of the bisphosphonate; wherein the pharmaceutical composition may further comprise an enhancer, which can be a medium chain fatty acid salt, an ester, an ether, or a derivative of a medium chain fatty acid and can have a carbon chain length of from about 4 to about 20 carbon atoms.

In some embodiments, the enhancer is a carboxylic acid or a salt thereof having a carbon chain length of from about 8 to about 12 carbon atoms. In some embodiments, enhancer is sodium decanoate.

In some embodiments, the ratio of the bisphosphonate, such as zoledronic acid, to the enhancer, such as sodium decanoate, is from about 1:5 to about 1:10, or about 1:25 to about 1:30. In some embodiments, a dosage form comprises about 10 mg to about 20 mg of zoledronic acid and about 500 mg to about 600 mg of sodium decanoate.

In some embodiments, a dosage form comprises about 20 mg of zoledronic acid, about 550 mg of sodium decanoate, about 275 mg of sorbitol, about 4.5 mg of colloidal silicon dioxide, about 45 mg of crospovidone, about 4.5 mg of stearic acid, about 54 mg of Opadry 1 yellow, about 81 mg of Acryl-EZE II, and about 1.3 mg of talc.

In some embodiments, the pain being treated is associated with arthritis, inflammatory pain, musculoskeletal pain, complex regional pain syndrome, neuropathic pain, or low back pain.

In some embodiments, the dosage form administered is Orazol®.

### DETAILED DESCRIPTION

It should be appreciated that any methods disclosed herein can be embodied in different forms and should not be construed as limited to the embodiments set forth herein. Rather, these embodiments are provided so that this disclosure will be thorough and complete, and will fully convey the scope of the intended methods to those skilled in the art.

An oral dosage form of a bisphosphonate described herein such as, but not limited to, zoledronic acid, may be used to treat, or provide relief of, any type of pain including, but not limited to, inflammatory pain, arthritis pain, complex regional pain syndrome, lumbosacral pain, musculoskeletal pain, neuropathic pain, chronic pain, cancer-related pain, acute pain, postoperative pain, etc. In some instances, pain relief may be palliative, or pain relief may be provided independent of improvement of the disease or condition or the underlying cause of the disease or condition. For example, although the underlying disease may not improve, or may continue to progress, an individual suffering from the disease may experience pain relief. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

In some embodiments, the mammal being treated is not suffering from bone metastasis. In some embodiments, the mammal being treated is not suffering from cancer. In some embodiments, the mammal being treated is not suffering from osteoporosis.

For example, zoledronic acid or another bisphosphonate may be administered orally to relieve musculoskeletal pain including lower back pain and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoporosis of the hip, vertebral crush fractures, osteoporosis, etc. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

A bisphosphonate, such as zoledronic acid, may also be used to treat bone fractures or to enhance the healing of bone fractures. In some embodiments, the bisphosphonate may be used to treat pain associated with bone fractures.

In some embodiments, zoledronic acid or another bisphosphonate may also be administered orally to relieve neuropathic pain, including diabetic peripheral neuropathy, post-herpetic neuralgia, trigeminal neuralgia, monoradiculopathies, phantom limb pain, and central pain. Other causes of neuropathic pain include cancer-related pain, lumbar nerve root compression, spinal cord injury, post-stroke pain, central multiple sclerosis pain, HIV-associated neuropathy, and radio-therapy or chemo-therapy associated neuropathy. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

In some embodiments, zoledronic acid or another bisphosphonate may be administered orally to relieve inflammatory pain including musculoskeletal pain, arthritis pain, and complex regional pain syndrome. In some embodiments, enhanced bioavailability of the zoledronic acid may be achieved in treating one of these conditions by administering a dosage form comprising zoledronic acid in the form of a disodium salt. This may allow a reduced molar amount of the disodium salt to be used as compared to what would be used with the diacid form.

Examples of musculoskeletal pain include low back pain; and pain associated with vertebral crush fractures, fibrous dysplasia, osteogenesis imperfecta, Paget's disease of bone, transient osteoporosis, and transient osteoporosis of the hip.

A bisphosphonate, such as zoledronic acid, may also be used to treat lower back pain, or other musculoskeletal or inflammatory conditions, having a change in bone that is detectable by MRI or another medical imaging instrument. For example, a bisphosphonate, such as zoledronic acid, may be used to treat lower back pain associated Modic changes, or vertebral endplate signal changes (VESC) and bone marrow changes visible using magnetic resonance imaging (MRI). Modic changes, can be classified into various types including type 1 (M1), type 2 (M2), and type 3 (M3) lesions or changes, any of which may be treated using a bisphosphonate such as zoledronic acid. VESCs may be found in patients with different types of low back pain including but not limited to spondylitis, trauma, spondyloarthropathies including ankylosing spondylitis, Schmorl's nodes, fracture, tumor, and spinal cord infarction. Lesions in ankylosing spondylitis include osteitis and spondylodiscitis, which can be detected using MRI or another medical imaging instrument.

The terms “treat”, “treatment”, “treating” as disclosed herein includes their common meaning in the field and includes reversing, alleviating, or inhibiting the progress of a medical condition, disorder or disease as described herein and also includes any medical treatment or application of medical aid applied in an effort to address the effects of an ailment, a medical condition and/or a pathology either directly or indirectly and includes treating pain associated with the ailment, the medical condition and/or the pathology in question. In some embodiments, the term may also include the treatment of any undesirable symptom. For example, in some embodiments, the medical condition to be treated may include pain.

As used herein, “a medical condition that is responsive to a bisphosphonate compound” refers to medical conditions that may be treated or prevented, or pain resulting from these conditions that may be relieved, by administering a bisphosphonate compound. In some embodiments, medical conditions include, but are not limited to, osteoporosis, rheumatoid arthritis, bone fracture, excessive bone resorption, and combinations thereof. In some embodiments, medical conditions include, but are not limited to, SLE, cancer (e.g., prostate cancer, metastatic bone cancer, lung cancer, multiple myeloma breast cancer, and any solid tumor that induces metastatic disease), tumor induced hypocalcemia, bone metastasis, and combinations thereof.

The terms “bisphosphonate”, as used herein, include acids, salts, esters, hydrates, polymorphs, hemihydrates, solvates, and derivatives of suitable bisphosphonate compounds. Non-limiting examples of bisphosphonates useful herein include the following:

(a) Alendronate, also known as Alendronic acid, 4-amino-1-hydroxybutylidene-1,1-bisphosphonic acid, alendronate sodium, alendronate monosodium trihydrate or 4-amino-1-hydroxybutylidene-1,1-bisphosphonic acid monosodium trihydrate;

(b) [(cycloheptylamino)-methylene]-bis-phosphonate (incadronate);

(c) (dichloromethylene)-bis-phosphonic acid (clodronic acid) and the disodium salt (clodronate);

(d) [1-hydroxy-3-(1-pyrrolidinyl)-propylidene]-bis-phosphonate (EB-1053);

(e) (1-hydroxyethylidene)-bis-phosphonate (etidronate);

(f) [1-hydroxy-3-(methylpentylamino)propylidene]-bis-phosphonate (ibandronate);

(g) (6-amino-1-hydroxyhexylidene)-bis-phosphonate (neridronate);

(h) [3-(dimethylamino)-1-hydroxypropylidene]-bis-phosphonate (olpadronate);

(i) (3-amino-1-hydroxypropylidene)-bis-phosphonate (pamidronate);

(j) [2-(2-pyridinyl)ethylidene]-bis-phosphonate (piridronate);

(k) [1-hydroxy-2-(3-pyridinyl)-ethylidene]-bis-phosphonate (risedronate);

(l) {[(4-chlorophenyl)thio]methylene}-bis-phosphonate (tiludronate),

(m) Zoledronate also known as zoledronic acid, 1-hydroxy-2-(1H-imidazol-1-yl)ethylidene]-bis-phosphonate (zoledronate); and

(n) [1-hydroxy-2-imidazopyridin-(1,2-a)-3-ylethylidene]-bis-phosphonate (minodronate).

In some embodiments, the bisphosphonate may be selected from risedronate, alendronate, pamidronate, tiludronate, cimadronate, ibandronate, clodronate, or zoledronate. In some embodiments, the bisphosphonate is zoledronic acid.

As used throughout this specification and claims, the term “zoledronate or zoledronic acid” includes the related bisphosphonic acid forms, pharmaceutically acceptable salt forms, and equilibrium mixtures of these. The term “zoledronate” includes crystalline, hydrated crystalline, and amorphous forms of zoledronate and pharmaceutically acceptable salts.

The term “bisphosphonates” include all forms thereof including stereoisomers, enantiomers, diastereomers, racemic mixtures and derivatives thereof, for example, salts, acids, esters, and the like. The bisphosphonate may be provided in any suitable phase state including as a solid, liquid, solution, suspension, and the like. When provided in solid particulate form, the particles may be of any suitable size or morphology and may assume one or more crystalline, semi-crystalline and/or amorphous forms.

Non-limiting examples of bisphosphonate salts useful herein include those selected from the group alkali metal (e.g. sodium, potassium etc), alkaline metal, ammonium, and mono-, di-, tri-, or tetra C₁-C₃₀ alkyl-substituted ammonium.

The bisphosphonates that may be used in the present disclosure are further discussed in the U.S. Application Publication Nos. 2003/0139378 and 2004/0157799, which are incorporated by reference in their entireties.

A bisphosphonate, such as zoledronic acid may also be used to treat pain resulting from osteoarthritis of the knee, such as osteoarthritis of the knee associated with bone marrow lesions (BML), including BML that may be detected using MRI or another medical imaging instrument. In some embodiments, a bisphosphonate, such as zoledronic acid, may be used to treat pain resulting from osteoarthritis of the knee associated with bone marrow edema (BME), including BME which may be detected using MRI or another medical imaging instrument.

Arthritis includes its common meaning in the field and includes inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropaties including Charcot's foot, axial spondyloarthritis including ankylosing spondylitis, and SAPHO syndrome.

In some embodiments, a human being that is treated for a disease or condition that results in pain, such as an inflammatory condition, e.g. arthritis, by an oral dosage form of zoledronic acid, has an age of about 10 years to about 90 years, about 20 years to about 80 years, about 30 years to about 75 years old, about 40 years to about 70 years, about 1 year to about 16 years, or about 80 years to about 95 years.

In some embodiments, a human being that is treated for pain caused by a disease or condition, such as an inflammatory condition, e.g. arthritis, by an oral dosage form of zoledronic acid, has suffered from the arthritis for at least 1 month, at least 2 months, at least 6 months, or at least 1 year. In some embodiments, the bisphosphonate can be effective at relieving pain associated with inflammatory conditions.

In some embodiments, the pain caused by the disease or condition, such as an inflammatory condition, e.g. arthritis, affects a knee, an elbow, a finger, a wrist, a shoulder, or a hip.

In some embodiments, zoledronic acid or another bisphosphonate may be administered orally to relieve pain associated with complex regional pain syndrome, such as complex regional pain syndrome type I (CRPS-I), complex regional pain syndrome type II (CRPS-II), CRPS-NOS, or another type of CRPS. CRPS is a type of inflammatory pain. CRPS can also have a neuropathic component.

Complex regional pain syndrome may be a debilitating pain syndrome. It may be characterized by severe pain in a limb accompanied by edema, and autonomic, motor and sensory changes.

With respect to use of oral zoledronic acid for relieving pain associated with an inflammatory condition, relief of pain can be short-term, e.g. for a period of hours after administration of the dosage form, and/or relief of pain can be long-term, e.g. lasting for days, weeks, or even months after oral administration of zoledronic acid. In some embodiments, a mammal, such as a human being, experiences significant pain relief at least about 3 hours, at least about 6 hours, at least about 12 hours, at least about 24 hours, at least about 48 hours, at least about one week, at least about 2 weeks, or at least about 3 weeks after administration of an oral dosage form comprising zoledronic acid. In some embodiments, a mammal, such as a human being, experiences significant pain relief during at least part of the time from about 3 hours to about 2 weeks, about 3 hours to about 3 weeks, about 3 hours to about 24 hours, about 6 hours to about 2 weeks, or about 6 hours to about 24 hours, about 3 days to about 2 weeks, about 6 days to about 2 weeks, after administration of an oral dosage form comprising zoledronic acid.

Zoledronic acid or another bisphosphonate may also be administered orally to relieve cancer-related pain, including pain associated with multiple myeloma and bone metastases from solid tumors. In some embodiments, zoledronic acid may be used to treat pain that is not cancer-related pain. For example, zoledronic acid may be used to treat pain that is not associated with multiple myeloma, bone metastasis from solid tumors, hypercalcemia of malignancy, giant cell tumor of bone, blood cancers or leukemias, or solid tumors or cancers.

In some embodiments, a bisphosphonate may be used to treat pain associated with any medical condition described herein.

In addition to relieving pain, oral administration of zoledronic acid or another bisphosphonate may also be useful to treat diseases or conditions that may or may not include a pain component. For example, zoledronic acid or another bisphosphonate may be useful to treat any of the pain conditions or types of conditions listed above, including treatment that does not simply relieve the pain of those conditions, and treatment that is carried out in such a way that the condition is treated without pain relief occurring. In addition to any pain relief that zoledronic acid or another bisphosphonate may or may not provide, zoledronic acid or another bisphosphonates may be used to treat a disease or condition such as a metabolic disease or condition; an inflammatory disease or condition, including an inflammatory disease or condition that is not associated with pain; a cancer disease or condition; a neurological disease or condition; etc.

In some embodiments, oral administration of zoledronic acid or another bisphosphonate may also be useful to treat complex regional pain syndrome, and pain associated with any of the following: rheumatoid arthritis, osteoarthritis, erosive osteoarthritis, axial spondyloarthritis including ankylosing spondylitis, acute vertebral crush fracture, fibrous dysplasia, SAPHO syndrome, osteoporosis, transient osteoporosis, or transient osteoporosis of the hip.

In some embodiments, oral administration of zoledronic acid or another bisphosphonate may also be useful to treat pain associated with hypercalcemia of malignancy, multiple myeloma, bone metastases from solid tumors, Paget's disease of bone, giant cell tumor of bone, blood cancers or leukemias, or solid tumors or cancers. In some embodiments, a bisphosphonate composition may be useful for treating pain associated with any of these ailments.

In some embodiments, the dosage for bisphosphonate therapy (e.g. zoledronic acid concentrate for intravenous infusion) for osteoporosis related conditions is about 10% of the dosage for oncology treatment. For the treatment of osteoporosis related conditions, the bisphosphonate may be administered 5 mg annually. For prevention of pain associated with osteoporosis related condition, the bisphosphonate may be administered as 5 mg every other year. For the treatment of pain associated with oncology related conditions, in some embodiments, the bisphosphonate may be administered 4 mg every four weeks.

Further disclosed herein are methods of treatment or prevention of a medical condition that is responsive to a bisphosphonate compound. The methods comprise administering to the subject a pharmaceutical composition comprising a therapeutically effective amount of the bisphosphonate, that in some embodiments, may be given no less frequently than a bi-weekly dosage schedule, or in some embodiments, a weekly or daily dosage schedule. In some embodiments, the bisphosphonate compound is zoledronate. In some embodiments, the bisphosphonate is orally administered to the subject. In some embodiments, the methods described herein provide sustained pain relieving therapeutic effects of the bisphosphonate. In some embodiments, the methods described herein provide reduced adverse effects resulting from administering a bisphosphonate compound to the subject.

In some embodiments, the medical conditions accompanied by pain are selected from osteoporosis, rheumatoid arthritis, bone fracture, excessive bone resorption and a combination thereof. In some embodiments, the medical conditions are selected from systemic lupus erythematosus (SLE), cancer, tumor induced hypocalcemia, bone metastasis and a combination thereof. In some embodiments, the cancer is selected from the group consisting of prostate cancer, metastatic bone cancer, lung cancer, multiple myeloma, breast cancer and any solid tumor that induces metastatic disease.

In some embodiments, the pharmaceutical composition may be in a solid oral dosage form. In some embodiments, the pharmaceutical composition further comprises an enhancer. In some embodiments, the enhancer may be a medium chain fatty acid salt, an ester, an ether, or a derivative of a medium chain fatty acid and has a carbon chain length of from about 4 to about 20 carbon atoms. In some embodiments, the carbon chain length of the enhancer may be from 6 to 20 or 8 to 14 carbon atoms. In some embodiments, the enhancer may be selected from the group consisting of sodium caprylate, sodium caprate, sodium laurate and a combination thereof. In some embodiments, the enhancer is sodium caprate. In some embodiments, the enhancer may lead to an increase in the effective pain relief experienced by the patient being treated. Unless the context indicates otherwise, it is specifically intended that the various features described herein can be used in any combination.

Moreover, the present disclosure also contemplates that in some embodiments, any feature or combination of features set forth herein can be excluded or omitted.

All patents, patent applications, and publications referred to herein are incorporated by reference in their entirety. In case of a conflict in terminology, the present disclosure is controlling.

In some embodiments, the bisphosphonate is administered to the subject via intravenous administration. In some embodiments, the bisphosphonate is orally administered to the subject.

In some embodiments, the treatment or prevention described herein may provide sustained therapeutic effects of the bisphosphonate. As used herein, “sustained therapeutic effect” refers to a relatively constant efficacy level of the bisphosphonate compound in the administered subject. In some embodiments, the sustained therapeutic effect is reflected by the relatively sustained level of the applicable biomarkers, for example, the fluctuations of the biomarkers is no more than about 5%, 10%, 20% or 30% of the mean level of the biomarkers during the treatment. As used herein, “during the treatment” is the period that the bisphosphonate is periodically administered to the subject. Any applicable biomarkers may be used in the present method, e.g., those biomarkers associated with bone metabolism. Exemplary biomarkers include, but are not limited to, bone alkaline phosphatase, N-Telopeptide Cross-Links (NTX) in urine, serum C-telopeptide (CTX), or serum calcium level.

In some embodiments, the bisphosphonate compound is administered to the subject for the treatment of pain resulting from any of the medical conditions disclosed herein.

In some embodiments, the methods described herein may provide reduced adverse effects resulting from administering a bisphosphonate compound to the subject. As used herein, “reduced adverse effects” refers to a reduction in frequency and/or severity of adverse effects compared to a bisphosphonate compound administered via a method commonly used in the market (e.g., IV infusion) on a monthly or yearly dosage schedule. The adverse effect may be any toxic or side effects resulting from administering the bisphosphonate compound. In some embodiments, the adverse effect is selected from renal damage, general malaise, acute phase reaction, stomach pain, fatigue, nausea, or a combination thereof. In some embodiments, the acute phase reaction is selected from fever, muscle pain, bone pain, or a combination thereof.

In some embodiments, the bisphosphonate can be administered to the subject on a weekly dosage schedule or a daily dosage schedule. In some embodiments, when the pharmaceutical composition is administered orally, the oral dose of the bisphosphonate compound is about 8 to 400 times or 8 to about 200 times more than the systemic dose of bisphosphonate compound administered through intravenous infusion. As used herein, “systemic dose” refers to the amount of a bisphosphonate compound delivered to the circulatory system of a subject via either intravenous infusion or oral administration. As used herein, “oral dose” refers to the amount of a bisphosphonate compound in an oral dosage form of the bisphosphonate compound, for example, the amount of the bisphosphonate compound in one or more tablets or capsules.

In some embodiments, the methods described herein may be used to treat or prevent pain resulting from osteoporosis related conditions such as osteoporosis, rheumatoid arthritis, bone fracture, excessive bone resorption or a combination thereof. When the methods described herein are used to treat osteoporosis related medical conditions, the systemic dose of the pharmaceutical composition is in a range of about 0.000018 mmol (e.g., 0.005 mg zoledronic acid) to about 0.00015 mmol (e.g., 0.04 mg zoledronic acid) of the bisphosphonate compound per day. In some embodiments, the systemic dose of the pharmaceutical composition is in a range of about 0.00013 mmol (e.g., 0.035 mg zoledronic acid) to about 0.001 mmol (e.g., 0.28 mg zoledronic acid) of the bisphosphonate compound per week. In some embodiments, when the bisphosphonate (e.g., zoledronic acid) is administered in a dosage form of a tablet on a weekly dosage schedule and the bioavailability of the tablet is about 5%, the oral dosage of the bisphosphonate compound is in a range of about 0.0026 mmol (e.g., 0.7 mg zoledronic acid) to about 0.02 (e.g., 5.6 mg zoledronic acid). In some embodiments, when the bisphosphonate (e.g., zoledronic acid) is administered in a dosage form of a tablet on a biweekly dosage schedule and the bioavailability of the tablet is about 5%, the oral dose of the bisphosphonate compound is in a range of about 0.005 mmol (e.g., 1.4 mg zoledronic acid) to about 0.04 (e.g., 11.2 mg zoledronic acid). In some embodiments, when the bisphosphonate (e.g., zoledronic acid) is administered in a dosage form of a tablet on a daily dosage schedule and the bioavailability of the tablet is about 5%, the oral dose of the bisphosphonate compound is in a range of about 0.00037 mmol (e.g., 0.1 mg zoledronic acid) to about 0.0028 (e.g., 0.8 mg zoledronic acid). The ranges provided herein are intended to provide exemplary ranges of the oral dose for bisphosphonate in a tablet dosage form. However, the oral dose may vary when the bioavailability of the tablet changes.

In some embodiments, an oral dosage form of a bisphosphonate (such as zoledronic acid) administered weekly comprises from about 10 mg to about 20 mg of the bisphosphonate. In some embodiments, a weekly administration of a bisphosphonate occurs for about three to about four consecutive weeks. In some embodiments, a bisphosphonate is administered at bed time after a four-hour fast or in the morning after an overnight fast and about 30 minutes before eating breakfast. In some embodiments, a bisphosphonate is administered after about ten hours of fasting, and food is not consumed until at least about four hours after administration. In some embodiments, a bisphosphonate is administered at bed time after about four hours of fasting, and food is not consumed the next day until at least about ten hours after administration. In some embodiments, a bisphosphonate is administered immediately before, with, or immediately after consuming food, and no food is then consumed for at least about four hours after administration. In some embodiments, a patient to whom a bisphosphonate is administered remains upright after administration for at least about 30 minutes, at least about 60 minutes, at least about two hours, or at least about four hours.

In some embodiments, the methods described herein are used to treat pain associated with oncology related conditions, for example, but are not limited to, systemic lupus erythematosus (SLE), cancer, tumor induced hypocalcemia, bone metastasis or a combination thereof. In some embodiments, the cancer may be any solid tumor that may induce bone metastatic diseases. In some embodiments, the cancer may be selected from prostate cancer, metastatic bone cancer, lung cancer, multiple myeloma, breast cancer and any solid tumor that induces metastatic disease. When the methods described herein are used to treat oncology related conditions, the systemic dose of the pharmaceutical composition may be in a range of about 0.00018 mmol (e.g., 0.05 mg zoledronic acid) to about 0.0015 mmol (e.g., 0.4 mg zoledronic acid) of the bisphosphonate compound per day. In some embodiments, the systemic dose of the pharmaceutical composition may be in a range of about 0.0013 mmol (e.g., 0.35 mg zoledronic acid) to about 0.01 mmol (e.g., 2.8 mg zoledronic acid) of the bisphosphonate compound per week. In some embodiments, when the bisphosphonate (e.g., zoledronic acid) is administered in a dosage form of a tablet on a weekly dosage schedule and the bioavailability of the tablet may be about 5%, the oral dosage of the bisphosphonate compound may be in a range of about 0.026 mmol (e.g., 7 mg zoledronic acid) to about 0.2 (e.g., 56 mg zoledronic acid). In some embodiments, when the bisphosphonate (e.g., zoledronic acid) is administered in a dosage form of a tablet on a biweekly dosage schedule and the bioavailability of the tablet may be about 5%, the oral dose of the bisphosphonate compound may be in a range of about 0.05 mmol (e.g., 14 mg zoledronic acid) to about 0.4 (e.g., 112 mg zoledronic acid). In some embodiments, when the bisphosphonate (e.g., zoledronic acid) is administered in a dosage form of a tablet on a daily dosage schedule and the bioavailability of the tablet may be about 5%, the oral dose of the bisphosphonate compound may be in a range of about 0.0037 mmol (e.g., 1 mg zoledronic acid) to about 0.028 (e.g., 8 mg zoledronic acid). The ranges provided herein are intended to provide exemplary ranges of the oral dosage for bisphosphonate in a tablet dosage form. However, the oral dosage may vary when the bioavailability of the tablet changes.

In some embodiments, when the pharmaceutical composition of the bisphosphonate compound is administered at the dosage schedule described herein, the sustained therapeutic effect and reduced adverse effects may be provided with or without the enhancers described herein and the pharmaceutical composition may be administered via any applicable administration methods.

It is understood that a specific dose level for any particular subject may depend upon a variety of factors including the activity of the specific bisphosphonate compound employed, the age, body weight, general health, sex, diet, time of administration, rate of excretion, drug combination, and the severity of the particular disease being treated and form of administration. It is further understood that the ordinarily skilled physician or veterinarian will readily determine and prescribe the effective amount of the bisphosphonate compound for prophylactic or therapeutic treatment of the condition for which treatment is administered.

The amount of bisphosphonate active ingredient contained in the oral dosage forms disclosed herein will depend on the particular bisphosphonate selected and the dosage schedule upon which the bisphosphonate is dosed to the patient. The dosage schedules of daily, weekly, and biweekly are non-limiting examples of dosage regimens suitable for use with the oral dosage forms or intravenous infusion. The term “biweekly” means that a dosage form is administered once every 14 days. The terms “weekly” means that a dosage form is administered once every 7 days. The term “daily” means that a dosage form is administered once every day.

As used herein, a “therapeutically effective amount” refers to an amount of a bisphosphonate that elicits a therapeutically useful response in treating an existing medical condition and/or preventing or delaying the onset of a medical condition from occurring in a subject. In some embodiments, the subject is a mammal. In some embodiments, the subject is a human.

In some embodiments, in the methods described herein, the bisphosphonate may be administered in an oral dosage form. In some embodiments, when the pharmaceutical composition is administered orally, the pharmaceutical composition may further comprise an enhancer. As used herein, the term “enhancer” refers to a compound (or a mixture of compounds) which is capable of enhancing the transport of a drug, such as a bisphosphonate compound, across the GI tract in a subject such as a human. In some embodiments, the enhancer is a medium chain fatty acid or a medium chain fatty acid derivative having a carbon chain length of from 4 to 20 carbon atoms, or 6 to 20 carbon atoms. In some embodiments, the enhancer is a medium chain fatty acid or a medium chain fatty acid derivative having a carbon chain length of from 6 to 20 carbon atoms with the provisos that (i) where the enhancer is an ester of a medium chain fatty acid, said chain length of from 6 to 20 carbon atoms relates to the chain length of the carboxylate moiety, and (ii) where the enhancer is an ether of a medium chain fatty acid, at least one alkoxy group has a carbon chain length of from 6 to 20 carbon atoms. In some embodiments, the enhancer is solid at room temperature and has a carbon chain length of from 8 to 14 carbon atoms. In some embodiments, the enhancer is a sodium salt of a medium chain fatty acid. In a further embodiment, the enhancer is sodium caprylate, sodium caprate, sodium laurate or a combination thereof. In some embodiments, the enhancer is sodium caprate. In some embodiments, the drug (bisphosphonate) and enhancer can be present in a ratio of from 1:100000 to 10:1 (drug (bisphosphonate):enhancer) or from 1:1000 to 10:1. The enhancers are further described in U.S. Pat. Nos., 7,658,938 and 7,670,626, and U.S. Patent Application Publication Nos. 2003/0091623 and 2007/0238707, which are incorporated by reference in their entirety.

As used herein, the term “medium chain fatty acid derivative” includes fatty acid salts, esters, ethers, acid halides, amides, anhydrides, carboxylate esters, nitrites, as well as glycerides such as mono-, di- or tri-glycerides. The carbon chain may be characterized by various degrees of saturation.

In some embodiments, the carbon chain may be fully saturated or partially unsaturated (i.e. containing one or more carbon-carbon multiple bonds). The term “medium chain fatty acid derivative” is referred to encompass also medium chain fatty acids wherein the end of the carbon chain opposite the acid group (or derivative) is also functionalized with one of the above mentioned moieties (i.e., an ester, ether, acid halide, amide, anhydride, carboxylate esters, nitrile, or glyceride moiety). Such difunctional fatty acid derivatives thus include for example diacids and diesters (the functional moieties being of the same kind) and also difunctional compounds comprising different functional moieties, such as amino acids and amino acid derivatives, for example a medium chain fatty acid or an ester or a salt thereof comprising an amide moiety at the opposite end of the fatty acid carbon chain to the acid or ester or salt thereof.

As used herein, a “therapeutically effective amount of an enhancer” refers to an amount of enhancer that enhances intestinal delivery of the drug such as a bisphosphonate compound to the underlying circulation and allows for the uptake of a therapeutically effective amount of the drug such as a bisphosphonate compound via oral administration. It has been shown that the effectiveness of an enhancer in enhancing the gastrointestinal delivery of poorly permeable drugs is dependent on the site of administration, the site of optimum delivery being dependent on the drug and enhancer.

In some embodiments, the enhancer can lead to an overall improvement of the pain relieving effects of the active ingredient.

The combination of bisphosphonates and enhancers is further described in U.S. Patent Application Publication No. 2007/0238707, 2010/0215743, and U.S. Pat. No. 7,704,977, all of which are incorporated by reference in their entirety.

In some embodiments, the pharmaceutical composition is in an oral dosage form, e.g., solid oral dosage form. The oral dosage form of bisphosphonates described herein may deliver an effective amount of bisphosphonates to a patient quickly and without any of the deleterious side effects associated with intravenous infusion.

In some embodiments, the oral dosage form may be a tablet, a multiparticulate, or a capsule. In some embodiments, the oral dosage form is a delayed release dosage form which minimizes the release of drug and enhancer in the stomach, and hence the dilution of the local enhancer concentration therein, and releases the drug and enhancer in the intestine. In some embodiments, the oral dosage form is a delayed release rapid onset dosage form. Such a dosage form minimizes the release of drug and enhancer in the stomach, and hence the dilution of the local enhancer concentration therein, but releases the drug and enhancer rapidly once the appropriate site in the intestine has been reached, maximizing the delivery of the poorly permeable drug by maximizing the local concentration of drug and enhancer at the site of absorption.

As used herein, the term “tablet” includes, but is not limited to, immediate release (IR) tablets, sustained release (SR) tablets, matrix tablets, multilayer tablets, multilayer matrix tablets, extended release tablets, delayed release tablets and pulsed release tablets any or all of which may optionally be coated with one or more coating materials, including polymer coating materials, such as enteric coatings, rate-controlling coatings, semi-permeable coatings and the like. In some embodiments, pain relieving effects can be enhanced or otherwise programmed based on the type of formulation of the tablet. The term “tablet” also includes osmotic delivery systems in which a drug compound such as a bisphosphonate is combined with an osmagent (and optionally other excipients) and coated with a semi-permeable membrane, the semi-permeable membrane defining an orifice through which the drug compound may be released. Tablet solid oral dosage forms of the pharmaceutical composition used herein include, but are not limited to, those selected from the group consisting of IR tablets, SR tablets, coated IR tablets, matrix tablets, coated matrix tablets, multilayer tablets, coated multilayer tablets, multilayer matrix tablets and coated multilayer matrix tablets. In some embodiments, the tablet dosage form is an enteric coated tablet dosage form. In some embodiments, the tablet dosage form is an enteric coated rapid onset tablet dosage form. In some embodiments, an enteric coating may lead to an increase pain relieving effect of the dosage form.

As used herein, the term “capsule” includes instant release capsules, sustained release capsules, coated instant release capsules, coated sustained release capsules, delayed release capsules and coated delayed release capsules. In some embodiments, the capsule dosage form is an enteric coated capsule dosage form. In some embodiments, the capsule dosage form is an enteric coated rapid onset capsule dosage form. In some embodiments, a capsule may lead to an increased pain relieving effect of the composition.

The term “multiparticulate” as used herein means a plurality of discrete particles, pellets, mini-tablets and mixtures or combinations thereof. If the oral form is a multiparticulate capsule, hard or soft capsule, e.g., gelatin capsules, can suitably be used to contain the multiparticulate. In some embodiments, a sachet can suitably be used to contain the multiparticulate. The multiparticulate may be coated with a layer containing rate controlling polymer material. The multiparticulate oral dosage form may comprise a blend of two or more populations of particles, pellets, or mini-tablets having different in vitro and/or in vivo release characteristics. For example, a multiparticulate oral dosage form may comprise a blend of an instant release component and a delayed release component contained in a suitable capsule. In some embodiments, the multiparticulate dosage form comprises a capsule containing delayed release rapid onset minitablets. In some embodiments, the multiparticulate dosage form comprises a delayed release capsule comprising instant release minitablets. In a further embodiment, the multiparticulate dosage form comprises a capsule comprising delayed release granules. In some embodiments, the multiparticulate dosage form comprises a delayed release capsule comprising instant release granules. In some embodiments, a multiparticulate oral dosage form may lead to an increase pain relieving effect of the composition.

In some embodiments, the multiparticulate together with one or more auxiliary excipient materials may be compressed into tablet form such as a single layer or multilayer tablet. In some embodiments, a multilayer tablet may comprise two layers containing the same or different levels of the same active ingredient having the same or different release characteristics. In some embodiments, a multilayer tablet may contain a different active ingredient in each layer. The tablet, either single layered or multilayered, can optionally be coated with a controlled release polymer so as to provide additional controlled release properties.

In some embodiments, a multilayer tablet of the pharmaceutical composition used herein is provided. In some embodiments, such a multilayer tablet may comprise a first layer containing a bisphosphonate and an enhancer in an instant release form and a second layer containing a bisphosphonate and an enhancer in a modified release form. As used herein, the term “modified release” includes sustained, delayed, or otherwise controlled release of a bisphosphonate upon administration to a patient. In some embodiments, a multilayer tablet may comprise a first layer containing a bisphosphonate and a second layer containing an enhancer. Each layer may independently comprise further excipients chosen to modify the release of the bisphosphonate and/or the enhancer. Thus the bisphosphonate and the enhancer may be released from the respective first and second layers at rates which are the same or different. Alternatively, each layer of the multilayer tablet may comprise both a bisphosphonate and enhancer in the same or different amounts.

In some embodiments, a multiparticulate of the pharmaceutical composition used herein is provided. The multiparticulate may comprise particles, pellets mini-tablets or combinations thereof, and the bisphosphonate and the enhancer may be contained in the same or different populations of particles, pellets or minitablets making up the multiparticulate. In some embodiments, multiparticulate, sachets and capsules such as hard or soft gelatin capsules may suitably be used to contain the multiparticulate. A multiparticulate dosage form may comprise a blend of two or more populations of particles, pellets or minitablets having different in vitro and/or in vivo release characteristics. For example, a multiparticulate dosage form may comprise a blend of an immediate release component and a delayed release component contained in a suitable capsule.

The drug can be included in nano- or microparticulate drug delivery systems in which the drug is, or is entrapped within, encapsulated by, attached to, or otherwise associated with, a nano- or microparticle.

In the case of any of the embodiments described herein, a controlled release coating may be applied to the final dosage form (capsule, tablet, multilayer tablet etc.). In some embodiments, the controlled release coating may comprise a rate controlling polymer material as defined below. The dissolution characteristics of such a coating material may be pH dependent or independent of pH.

As used herein, the term “rate controlling polymer material” includes hydrophilic polymers, hydrophobic polymers, and mixtures of hydrophilic and/or hydrophobic polymers that are capable of controlling or retarding the release of the drug compound from a solid oral dosage form. Suitable rate controlling polymer materials include those selected from the group consisting of hydroxyalkyl cellulose such as hydroxypropyl cellulose and hydroxypropyl methyl cellulose; poly(ethylene) oxide; alkyl cellulose such as ethyl cellulose and methyl cellulose; carboxymethyl cellulose, hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate); and poly(vinyl acetate). Other suitable hydrophobic polymers include polymers and/or copolymers derived from acrylic or methacrylic acid and their respective esters, zein, waxes, shellac and hydrogenated vegetable oils.

In some embodiments, polymers included may be poly acrylic acid, poly acrylate, poly methacrylic acid and poly methacrylate polymers such as those sold under the Eudragit® trade name (Rohm GmbH, Darmstadt, Germany) specifically Eudragit® L, Eudragit® S, Eudragit® RL, Eudragit® RS coating materials and mixtures thereof. Some of these polymers can be used as delayed release polymers to control the site where the drug is released. They include polymethacrylate polymers such as those sold under the Eudragit™ trade name (Rohm GmbH, Darmstadt, Germany) specifically Eudragit® L, Eudragit® S, Eudragit® RL, Eudragit® RS coating materials and mixtures thereof.

The various embodiments of the oral dosage forms of the pharmaceutical composition disclosed herein may further comprise auxiliary excipient materials such as, for example, diluents, lubricants, disintegrants, plasticizers, anti-tack agents, opacifying agents, pigments, flavorings and the like. As will be appreciated by those skilled in the art, the exact choice of excipients and their relative amounts will depend to some extent on the final dosage form.

Suitable diluents include, for example, pharmaceutically acceptable inert fillers such as microcrystalline cellulose, lactose, dibasic calcium phosphate, saccharides, and/or mixtures of any of the foregoing. Examples of diluents include microcrystalline cellulose such as that sold under the Avicel trademark (FMC Corp., Philadelphia, Pa.) for example Avicel™ pH101, Avicel™ pH102 and Avicel™ pH112; lactose such as lactose monohydrate, lactose anhydrous and Pharmatose DCL21; dibasic calcium phosphate such as Emcompress® (JRS Pharma, Patterson, N.Y.); mannitol; starch; sorbitol; sucrose; and glucose.

Suitable lubricants, including agents that act on the flowability of the powder to be compressed, include, for example, colloidal silicon dioxide such as Aerosil™ 200; talc; stearic acid, magnesium stearate, and calcium stearate.

Suitable disintegrants include, for example, lightly cross-linked polyvinyl pyrrolidone, corn starch, potato starch, maize starch and modified starches, croscarmellose sodium, cross-povidone, sodium starch glycolate, and combinations and mixtures thereof.

The weight and size of oral dosage form may be adjusted to meet required systemic doses based on the percent of bioavailability of the bisphosphonate compound in the oral dosage form. Techniques for making these dose adjustments are known to those of skill in the art.

Some embodiments provide pharmaceutical formulations that comprise zoledronic acid, sodium decanoate, sorbitol, colloidal silicon dioxide, stearic acid, hydroxypropyl methylcellulose (e.g., opadry 1 yellow), enteric coating (e.g., Acryl-EZE II) and Talc. In some embodiments, the formulation is in a tablet dosage form.

The following embodiments are specifically contemplated.

### Embodiment 1

A method of treating pain, comprising administering a pharmaceutical composition for oral administration to a mammal in need thereof, wherein the pharmaceutical composition is effective in delivering therapeutically effective amounts of a drug and an enhancer to an intestine, said composition comprising zoledronic acid and an enhancer, wherein the composition is in a dosage form comprising about 1 mg to about 25 mg zoledronic acid, and wherein the enhancer is a medium chain fatty acid or a salt of a medium chain fatty acid having a carbon chain length of from 6 to 20 carbon atoms, is solid at room temperature, and is the only enhancer present in the composition.

### Embodiment 2

A method of treating pain, comprising administering solid oral dosage form to a mammal in need thereof, wherein the solid oral dosage form is effective in delivering therapeutically effective amounts of zoledronic acid and an enhancer to an intestine, said composition comprising zoledronic acid and an enhancer, wherein the enhancer is a medium chain fatty acid or a salt of a medium chain fatty acid having a carbon chain length of from 6 to 20 carbon atoms, is solid at room temperature, and is the only enhancer present in the composition, and wherein upon oral delivery of the composition to a human subject, the zoledronic acid has a bioavailability of 2.5% to 13.0%.

### Embodiment 3

The composition of embodiment 1 or 2, wherein the carbon chain length is from 8 to 14 carbon atoms.

### Embodiment 4

The composition of embodiment 1 or 2, wherein the enhancer is a sodium salt of a medium chain fatty acid.

### Embodiment 5

The composition of embodiment 4, wherein the enhancer is selected from the group consisting of sodium caprylate, sodium caprate, and sodium laurate.

### Embodiment 6

The composition of embodiment 1 or 2, wherein the drug and the enhancer are present in a ratio of from 1:100,000 to 10:1 (drug:enhancer).

### Embodiment 7

The composition of embodiment 1 or 2, further comprising at least one auxiliary excipient.

### Embodiment 8

The method of embodiment 1 or 2, wherein the zoledronic acid and the enhancer are administered in a solid oral dosage form having each of the zoledronic acid and the enhancer present in therapeutically effective amounts.

### Embodiment 9

The method of embodiment 8, wherein the dosage form is a tablet, a capsule, or a multiparticulate.

### Embodiment 10

The method of embodiment 8, wherein the dosage form is a delayed release dosage form.

### Embodiment 11

The method of embodiment 8, wherein the dosage form is a tablet.

### Embodiment 12

The method of embodiment 11, wherein the tablet is a multilayer tablet.

### Embodiment 13

The method of embodiment 8, wherein the dosage form further comprises a rate-controlling polymer material.

### Embodiment 14

The method of embodiment 13, wherein the rate-controlling polymer material is hydroxypropyl methyl cellulose.

### Embodiment 15

The method of embodiment 13, wherein the rate-controlling polymer material is a polymer derived from acrylic or methacrylic acid and their respective esters or copolymers derived from acrylic or methacrylic acid and their respective esters.

### Embodiment 16

The method of embodiment 13, wherein the composition is compressed into a tablet form prior to coating with the rate-controlling polymer material.

### Embodiment 17

The method of embodiment 16, wherein the tablet is a multilayer tablet.

### Embodiment 18

The method of embodiment 8, wherein the dosage form is a multiparticulate.

### Embodiment 19

The method of embodiment 18, wherein the multiparticulate comprises discrete particles, pellets, minitablets, or combinations thereof.

### Embodiment 20

The method of embodiment 19, wherein the multiparticulate comprises a blend of two or more populations of particles, pellets, minitablets, or combinations thereof each population having different in vitro or in vivo release characteristics.

### Embodiment 21

The method of embodiment 18, wherein the multiparticulate is encapsulated in a gelatin capsule.

### Embodiment 22

The method of embodiment 21, wherein the capsule is coated with a rate-controlling polymer material.

### Embodiment 23

The method of embodiment 18, wherein the multiparticulate is incorporated into a sachet.

### Embodiment 24

The method of embodiment 19, wherein the discrete particles, pellets, minitablets, or combinations thereof are compressed into a tablet.

### Embodiment 25

The method of embodiment 24, wherein the tablet is coated with a rate controlling polymer material.

### Embodiment 26

The method of embodiment 24, wherein the tablet is a multilayer tablet.

### Embodiment 27

The method of embodiment 25, wherein the tablet is a multilayer tablet.

### Embodiment 28

The method of embodiment 8, wherein the zoledronic acid and the enhancer are present in the dosage form in a ratio of from 1:100,000 to 10:1 (drug:enhancer).

### Embodiment 29

The method of embodiment 28, wherein the ratio is from 1:1,000 to 10:1 (drug:enhancer).

### Embodiment 30

The method of embodiment 8, wherein the composition is in the form of a delayed release enteric coated tablet.

### Embodiment 31

The method of embodiment 30, wherein the zoledronic acid and the enhancer are present in the dosage form in a ratio of from 1:1,000 to 10:1 (drug:enhancer).

### Embodiment 32

The method of embodiment 30, wherein the enhancer is sodium caprate.

### Embodiment 33

The method of embodiment 2, comprising about 1 mg to about 25 mg zoledronic acid.

### Embodiment 34

A method of treating or preventing pain in a subject, the method comprising: administering to the subject a pharmaceutical composition comprising a therapeutically effective amount of the bisphosphonate no less frequently than a bi-weekly dosage schedule, wherein the bisphosphonate compound is zoledronic acid.

### Embodiment 35

The method of embodiment 34, wherein the bisphosphonate is administered to the subject via intravenous administration.

### Embodiment 36

The method of embodiment 34, wherein the bisphosphonate is orally administered to the subject.

### Embodiment 37

The method of embodiment 34, wherein the treatment or prevention provides sustained therapeutic effects of the bisphosphonate.

### Embodiment 38

The method of embodiment 34, wherein the treatment or prevention provides reduced adverse effects resulting from administering a bisphosphonate compound to the subject comparing to the treatment of administering bisphosphonate compound via IV infusion or orally administration on a monthly or yearly dosage schedule.

### Embodiment 39

The method of embodiment 34, wherein the bisphosphonate is administered to the subject on a weekly dosage schedule.

### Embodiment 40

The method of embodiment 34, wherein the bisphosphonate is administered to the subject on a daily dosage schedule.

### Embodiment 41

The method of embodiment 34, wherein the pharmaceutical composition is administered orally, and the oral dose of the bisphosphonate compound is about 8 to 400 times more than the systemic dose of bisphosphonate compound administered through intravenous infusion.

### Embodiment 42

The method of embodiment 34, wherein the systemic dose of the pharmaceutical composition is in a range of about 0.000018 mmol to about 0.00015 mmol of the bisphosphonate compound per day.

### Embodiment 43

The method of embodiment 34, wherein the systemic dose of the pharmaceutical composition is in a range of about 0.00013 mmol to about 0.001 mmol of the bisphosphonate compound per week.

### Embodiment 44

The method of embodiment 34, wherein the pharmaceutical composition is in a solid oral dosage form.

### Embodiment 45

The method of embodiment 34, wherein the pharmaceutical composition further comprises an enhancer, wherein said enhancer is a medium chain fatty acid salt, an ester, an ether, or a derivative of a medium chain fatty acid and has a carbon chain length of from about 4 to about 20 carbon atoms.

### Embodiment 46

The method of embodiment 45, wherein the carbon chain length of the enhancer is from 6 to 20 carbon atoms.

### Embodiment 47

The method of embodiment 45, wherein the carbon chain length is from 8 to 14 carbon atoms.

### Embodiment 48

The method of embodiment 45, wherein the enhancer is a sodium salt of a medium chain fatty acid.

### Embodiment 49

The method of embodiment 45, wherein the enhancer is selected from the group consisting of sodium caprylate, sodium caprate, sodium laurate and a combination thereof.

### Embodiment 50

The method of embodiment 45, wherein the enhancer is sodium caprate.

### Embodiment 51

The method of embodiment 45, wherein the bisphosphonate and the enhancer are present in a ratio of from 1:100,000 to 10:1 (bisphosphonate:enhancer).

### Embodiment 52

The method of embodiment 45, wherein the composition is in the form of a delayed release enteric coated tablet.

The foregoing is illustrative and is not to be construed as limiting thereof. Although a few exemplary embodiments have been described, those skilled in the art will readily appreciate that many modifications are possible in the exemplary embodiments without materially departing from the novel teachings and advantages disclosed herein. Accordingly, all such modifications are intended to be included within the scope of this disclosure as defined in the claims. Therefore, it is to be understood that the foregoing is illustrative and is not to be construed as limited to the specific embodiments disclosed, and that modifications to the disclosed embodiments, as well as other embodiments, are intended to be included within the scope of the appended claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of skill in the art to which this disclosure belongs.

The term “consists essentially of” (and grammatical variants), as applied to the compositions of this disclosure, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components. The terms “a,” “an,” “the” and similar referents used in the context of describing various embodiments herein (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate various embodiments of the present disclosure and does not pose a limitation on the scope of any claim. No language in the specification should be construed as indicating any non-claimed element essential to the practice of the teachings of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience. When any such inclusion or deletion occurs, the specification is deemed to contain the group as modified thus fulfilling the written description of all Markush groups used in the appended claims.

Certain embodiments are described herein, including the best mode known to the inventor for carrying out the teachings of the present disclosure. Of course, variations on these described embodiments will become apparent to those of ordinary skill in the art upon reading the foregoing description. The inventor expects skilled artisans to employ such variations as appropriate, and the inventor intends for the teachings of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating or preventing pain caused by a medical condition in a subject, the method comprising:
administering to the subject a pharmaceutical composition comprising a therapeutically effective amount of zoledronic acid or a salt thereof;
wherein the pharmaceutical composition further comprises an enhancer selected from the group consisting of a medium chain fatty acid salt, an ester, an ether, or a derivative of a medium chain fatty acid.

2. The method of claim 1, wherein the enhancer has a carbon chain length of from about 8 to about 12 carbon atoms.

3. The method of claim 1, wherein the pharmaceutical composition is in a solid oral dosage form.

4. The method of claim 1, wherein the enhancer is sodium decanoate.

5. The method of claim 1, wherein the ratio of the zoledronic acid to the enhancer is from about 1:5 to about 1:10.

6. The method of claim 1, wherein the composition is in the form of a delayed release enteric coated tablet.

7. The method of claim 1, wherein the composition is in the form of an immediate release enteric coated tablet.

8. The method claim 1, wherein the composition comprises about 10 mg to about 20 mg of zoledronic acid and about 500 mg to about 600 mg of sodium decanoate.

9. The method of claim 8, wherein the pain is associated with arthritis.

10. The method of claim 8, wherein the pain is inflammatory pain.

11. The method of claim 8, wherein the pain is musculoskeletal pain.

12. The method of claim 8, wherein the pain is associated with complex regional pain syndrome.

13. The method of claim 8, wherein the pain is neuropathic pain.

14. The method of claim 8, wherein the pain is low back pain.

15. The method of claim 1, wherein the composition comprises about 20 mg of zoledronic acid, about 550 mg of sodium decanoate, about 275 mg of sorbitol, about 4.5 mg of colloidal silicon dioxide, about 45 mg of crospovidone, about 4.5 mg of stearic acid, about 54 mg of Opadry 1 yellow, about 81 mg of Acryl-EZE II, and about 1.3 mg of talc.

16. The method of claim 15, wherein the pain is associated with arthritis.

17. The method of claim 15, wherein the pain is inflammatory pain.

18. The method of claim 15, wherein the pain is musculoskeletal pain.

19. The method of claim 15, wherein the pain is associated with complex regional pain syndrome.

20. The method of claim 15, wherein the pain is low back pain.

21. A method of treating pain, comprising administering a pharmaceutical composition for oral administration to a mammal in need thereof, wherein the pharmaceutical composition is effective in delivering therapeutically effective amounts of a drug and an enhancer to an intestine, said composition comprising zoledronic acid and an enhancer, wherein the composition is in a dosage form comprising about 1 mg to about 25 mg zoledronic acid, and wherein the enhancer is a medium chain fatty acid or a salt of a medium chain fatty acid having a carbon chain length of from 6 to 20 carbon atoms, is solid at room temperature, and is the only enhancer present in the composition.

22. A method of treating pain, comprising administering solid oral dosage form to a mammal in need thereof, wherein the solid oral dosage form is effective in delivering therapeutically effective amounts of zoledronic acid and an enhancer to an intestine, said composition comprising zoledronic acid and an enhancer, wherein the enhancer is a medium chain fatty acid or a salt of a medium chain fatty acid having a carbon chain length of from 6 to 20 carbon atoms, is solid at room temperature, and is the only enhancer present in the composition, and wherein upon oral delivery of the composition to a human subject.
